(19) United States
(12) Patent Application Publication
(10) Pub. No.: US 2008/0169238 A1
(43) Pub. Date: Jul. 17, 2008

(54) BIOSORPTION SYSTEM PRODUCED FROM BIOFILMS SUPPORTED IN FAUJASITE (FAU) ZEOLITE, PROCESS OBTAINING IT AND ITS USAGE FOR REMOVAL OF HEXAVALENT CHROMIUM (CR(VI))

(75) Inventors: Maria Teresa JESUS SIMOES CAMPOS TAVARES, Braga (PT); Maria Isabel Pontes Correia Neves, Braga (PT)

Correspondence Address: SUGHRUE MION, PLLC, 2100 PENNSYLVANIA AVENUE, N.W., SUITE 800, WASHINGTON, DC, 20037, US

(73) Assignee: UNIVERSIDADE DO MINHO, Braga (PT)

(21) Appl. No.: 12/029,057

(22) Filed: Feb. 11, 2008

Related U.S. Application Data
(63) Continuation of application No. PCT/IB2006/052792, filed on Aug. 11, 2006.

(30) Foreign Application Priority Data
Aug. 12, 2005 (PT) 103332

(51) Int. Cl. C12N 11/14; C02F 3/34

(52) U.S. Cl. 210/616; 435/176

(57) ABSTRACT

The present invention refers to a biosorption system composed of a bacterial biofilm supported in synthetic zeolites, for usage in various types of industry for the removal of hexavalent chromium, through the retention of metal ions in the biofilm, in solutions with concentrations between 50 and 250 mg_{Cr}/L, process for obtaining it and respective usages. This process consists in obtaining a bacterial biofilm of Arthrobacter viscosus, supported on a faujasite (FAU) zeolite. The biofilm promotes the reduction of Cr (VI) to Cr (III) and, subsequently, Cr (III) is fixed in the zeolite by ion exchange. Several characterization procedures, like spectroscopic techniques (FTIR and ICP-AES), surface analysis (XRD and SEM) and thermal analysis (TGA) reveal that the biosorption process does not modify the morphology or the structure of the FAU zeolite. The biosorption system, and respective fixation process of hexavalent chromium in faujasite (FAU) zeolites, may be applicable to the treatment of industrial, mining or agriculture wastewater, for hexavalent chromium removal.

This application is a Continuation Application of International Application No. PCT/IB2006/052792 filed on Aug. 11, 2006, claiming priority based on Portugal Patent Application No. 103332, filed Aug. 12, 2005, the contents of all of which are incorporated herein by reference in their entirety.

### BACKGROUND OF THE INVENTION

1. Field of the Invention

The present invention is included within the domain of liquid effluents treatment, like for example the treatment of industrial, mining or agriculture wastewater by means of the removal of hexavalent chromium through its fixation in a faujasite zeolite.

2. Summary of the Invention

The present invention refers to a biosorption system composed of a bacterial biofilm supported on synthetic zeolites, for usage in various types of industry for the removal of hexavalent chromium, through the retention of metal ions in the biofilm, in solutions with concentrations between 50 and 250 mg_{Cr}/L, process for obtaining it and respective usages.

This process consists in obtaining a bacterial biofilm of Arthrobacter viscosus, supported on a faujasite (FAU) zeolite.

The biofilm promotes the reduction of Cr (VI) to Cr (III) and, subsequently, Cr (III) is fixed in the zeolite by ion exchange.

Several characterization procedures like spectroscopic techniques (FTIR and ICP-AES), surface analysis (XRD and SEM) and thermal analysis (TGA) reveal that the biosorption process does not modify the morphology or the structure of the FAU zeolite.

This process defines a low cost technology, appropriate for locally very active small companies producing liquid effluents with low heavy metal concentrations.

### BACKGROUND OF THE INVENTION

Nowadays, pollution control is one of the major concerns of the scientific and environmentalist communities. The main pollutants are the gases responsible for greenhouse effect, heavy metals, organic and inorganic effluents, and volatile organic compounds, VOC's. Chromium (Cr), in particular, is a toxic pollutant present in wastewater of many industries, as for example metallurgies, mining installations and tanning facilities. The soils are contaminated and, as a consequence, water sources and groundwater. There are numerous processes for removal of heavy metals in liquid solution, as for example chemical precipitation, ion exchange, membrane filtration, reverse osmosis and activated carbon adsorption [1]. These processes are quite expensive and not really efficient to be applied to low concentration solutions, so that small industries, with tight budgets, may have some difficulties in the accomplishment of environmental legislation regarding the emission of liquids.

Activated carbon adsorption is considered a competitive and efficient process for heavy metal removal in low concentrations. Nevertheless, the utilization of activated carbon is quite expensive and alternative sorbents and technologies are needed [2, 3]. In this perspective, the zeolites present a great potential for heavy metals removal from industrial wastewater. The zeolites are composed of SiO₄ and AlO₄ tetrahedrons bond by the oxygen atoms of the vertexes. The existence of a negative structural charge, due to the AlO₄⁵⁻ tetrahedrons, promotes a strong affinity for metal ions, defining the sorbent properties to these supports. Sodium, potassium and other positively charged exchangeable ions occupy the defined channels within the three-dimensional structure and can be replaced by heavy metals [4].

Several research works presented in the last years, refer the utilization of natural zeolites in effluent treatment. The removal of heavy metals from wastewater was studied, using clinoptilolite, the most abundant natural zeolite. The results for ion exchanged ranged between 1.6 mg/g for Pb²⁺ and 0.0 mg/g for Cr³⁺ [5]. This issue is complicated when the ion is Cr (VI), as it appears in chromate or dichromate form, i.e. negatively charged and with a high characteristic dimension, as it is normally quite hydrated. One way to solve the problem of Cr⁶⁺ removal in solution, is the combination of bacterial biosorption with the ion exchange ability of the zeolite.

Biosorption is the fixation of metals through biological materials without active uptake and can be considered as a collective term for a number of passive accumulation processes, which may include ion exchange, coordination, complexation, adsorption and microprecipitation [6]. Other authors [7] refer that biosorption is the ability of biological materials to accumulate heavy metals from waste streams by either metabolically mediation or by purely physical-chemical uptake pathways.

Bacteria are quite adequate for biosorption due to their ability to fix metal ions, adaptability to natural environments and low cost. Arthrobacter viscosus is a good exopolysaccharide producer, which, by itself, would allow foreseeing good qualities for support adhesion and for metal ions entrapment [8]. The system, described herewith, combine the biosorption properties of the microorganism with the characteristics of the zeolites, such as ion exchange ability and shape selectivity.

Among the heavy metals that may be removed from liquid solutions by biosorption, chromium demands special attention due to its several oxidation states. The process herein described allows the removal of chromium from K₂Cr₂O₇ liquid solutions with different initial concentrations. A reduction of Cr₂O₇²⁻ is performed by the biofilm. The metabolic reduction has been studied and modeled for different pure bacterial cultures [9]. The Arthrobacter viscosus bacterium supported on the zeolite reduces Cr (VI) to Cr (III) and the Cr (III) is retained in the zeolite by ion exchange.

Recent documents refer the utilization of biosorbents in the removal of heavy metals of effluents, as for example the usage of ceramic substrates covered with chitosan (US2003150802). However, chitosan is not efficient in Cr removal. FAU zeolites have been used as support for adsorption, mainly for separation of compounds in gaseous effluents, like car exhaust (JP2000202282, JP9192486, EP1356862, and U.S. Pat. No. 6,350,428, among others), but their application as a biofilm support for chromium removal is not known. Document US2004124150, “Hexavalent chromium removal from aqueous media using ferrous-form zeolite materials” suggest the removal of Cr through the contact with the zeolite, but does not take the biological path and does not offer a solution for the used zeolite.

Document “Lameiras et al.—Baterial biofilm supported on granular activated carbon and on natural zeolites—an application to wastewater treatment. CHISA Int. Cong. Chem. Process Eng. 2004, pp. 2929-2931” discloses biological systems for wastewater treatment., namely for chromium (VI) removal. These systems comprise an Arthrobacter viscosus biofilm supported on either granulated activated carbon or natural zeolites. The aim of this study is the comparison of the adsorption processes of metal ions in different supports covered by a biofilm, being this biofilm used only as a mean to entrap the metal ions from the liquid solution, but its ability to chemically reduce ions was not understood or even referred. The present invention discloses a biosorption system comprising a bacterial biofilm with a faujasite zeolite indicating that such biofilm would entrap the hexavalent ion (negatively charged) and its adsorption would occur in this oxidation state.

In the present invention, the selection of the support was optimized considering the combination of the reducing process [Cr(VI), negatively charged, is reduced to Cr(III), positively charged] performed by the biofilm, followed by an ion-exchange process in the faujasite, selected for its ability to exchange ions. The selection of these particular synthetic zeolites among multiple possibilities in terms of ion-exchangers, was based on the recent knowledge of the reducing action of the biofilm, without which their application was pointless, as faujasites do not adsorb negative ions.

Document EP0607636 describes the use of activated carbon as a support for bacterial films to remove organic compounds from the waste streams, which is quite different from what is described in the present invention. Document GB2312893 describes a_composition that includes a zeolite, potassium carbonate, hydrogen sulphate and denitrificating bacteria. This composition is suitable for water softening, pH control and algae growth control, which is also quite different from what is described in the present invention. None of those documents refers the use of faujasite zeolites, which reveal to be the most adequate choice to remove Cr (VI) ions from wastewaters due to its ion exchange ability associated to the biofilm reducing action.

The present invention aims solving the problem of chromate and dichromate contaminated water, compensating the gaps regarding current solutions for hexavalent chromium removal from effluents by presenting a biofilm supported on faujasite zeolites.

### DESCRIPTION OF THE INVENTION

The present invention proposes a biosorption system consisting of a biofilm of Arthrobacter viscosus supported on a FAU zeolite for removing heavy metal ions from current environment pollutants.

The FAU presents a negative structural charge matrix revealing a strong affinity for metal cations, conferring excellent adsorption properties to these supports. Positively charged exchangeable ions, as for example Na and K, occupy the channels within the three-dimensional structure, which can be replaced by heavy metals.

On the other hand, bacteria are quite adequate for heavy metals biosorption since they possess good capacity for fixing metal ions. The exopolysaccharide producer Arthrobacter viscosus, in particular, reveals excellent qualities for support adhesion and for metal ions entrapment, conferring to the biofilm—zeolite system properties, such as ion exchange and shape selectivity.

Therefore, dichromate ions are entrapped by the supported biofilm, starting from K₂Cr₂O₇ solutions of low concentration (concentrations between 50 mg_{Cr}/L and 250 mg_{Cr}/L). The zeolite presents a high surface area (500-700 m²/g⁻¹), but most of it is internal. The novelty of this method resides in the fact that the Arthrobacter viscosus bacteria, supported in the zeolite, reduces Cr (VI) to Cr (III), allowing a subsequent fixation of the cation inside the matrix. The resulting structure reveals highly adequate for application in environmental catalysis due to its activity and selectivity.

It is foreseen that this pollution control method may have a highly positive cost effective component, since the final product, that is, the matrix loaded with the metallic ion, presents adequate activity and selectivity for its application in environmental catalysis. In this way, the final product of this process of Cr (VI) removal, may be reintroduced in the industrial process instead of being released in the environment. Reference is made to the fact that the catalysts promoting the total oxidation of the volatile organic compounds, VOC, are defined in two categories: transition metals oxides (usually chromium, cobalt, copper, nickel and manganese) or noble metals (typically platinum or palladium). The high cost of noble metals promotes the interest regarding their substitution by transition metals. Therefore, the final product of the remediation process proposed herewith, opposing to the products obtained due precipitation and other classical treatment of effluents, reveals a strong potential for recycling with an ecological applicability.

This process defines a low cost technology appropriate for locally very active small companies, producing liquid effluents with low heavy metal concentrations. These companies have tight budgets and are not especially motivated in treating their wastes with the conventional pollution reducing technology, because of the high cost involved and for not being always efficient when applied to low concentration effluents.

### DETAILED DESCRIPTION OF THE INVENTION

The preparation of the biosorption system is carried out in such a way, that the FAU zeolites form a support matrix to the biofilm of Arthrobacter viscosus, retaining Cr ions, through inoculation in culture medium containing the mentioned bacteria, in batch experiments, and by adsorption of Cr ions to this support, according to the following:

1. Preparation of the Faujasite (FAU) Zeolites

The zeolites were obtained from W.R. Grace and were, prior to use, calcined at 500° C. during 8 hours under a dry air stream.

2. Preparation of the Biofilm Supported in FAU Zeolites

The preparation of the biosorbent is carried out trough the growing of the Arthrobacter viscosus bacteria in presence of pre-treated zeolites.

The Arthrobacter viscosus bacteria was obtained from the Spanish Type Culture Collection of the University of Valencia.

For the microorganism growth, a medium with 5 g/L of peptone, 3 g/L of malt extract, 3 g/L of yeast extract and 10 g/L of glucose is prepared, previously sterilized at 120° C. for 20 min. The Erlenmeyer flasks are kept at 28° C., in moderate stirring.

3. Adsorption Tests of Cr in the Biofilm-Zeolite

Adsorption tests are carried out, using 1.0 g of the NaY or NaX zeolite with 150 mL of the different dichromate solutions with a concentration between 50 and 250 mg_{Cr}/L and 15 mL of Arthrobacter viscosus culture medium in 250 mL Erlenmeyer flasks at 28° C., in moderate stirring.

Aqueous chromium solutions were prepared by dilution of K₂Cr₂O₇ (Aldrich) in distilled water.

The matrix obtained through centrifugation at 5000 rpm, is subsequently calcined at 500° C. during 6 hours under a dry air stream in order to remove the organic matter of the Arthrobacter viscosus bacterium, for a later catalytical utilization.

The biosorption system was tested under conditions of low Cr concentration, samples (1 mL) were systematically taken, and, after centrifugation, analyzed by atomic absorption spectrophotometry (AAS) for metal determination.

The material obtained in this way, after the biosorption process, is identified by the designation Crₙzeolite, where n represents the initial concentration of chromium in the solution to be treated.

The total chemical analysis of the zeolite, after biosorption, quantifies the entrapped metal.

The obtained data show a maximum efficiency of Cr removal of about 20%, realized through the usage of the described biosorption systems.

TABLE 1
Chemical analysis of the zeolite samples

| | Si/Al | Cr content (total w %) | Metal quant./cel. unit |
|---|---|---|---|
| NaY | 2.88 | — | — |
| NaX | 1.63 | — | — |
| Cr₁₀₀—NaY | 2.88 | 0.14 | 0.45 |
| Cr₁₀₀—NaX | 1.63 | 0.19 | 0.67 |

No significant variations were found in the diffraction patterns relatively to the structure of the NaY and NaX zeolites, before and after the biosorption.

Through thermal analysis (TGA) was verified that the biosorption systems of zeolites, containing the biofilm, present the same type of performance than the original support.

Data obtained trough spectroscopy FTIR show that in the systems, spectra are dominated by strong zeolite bands, namely from 3,700-3,300 cm⁻¹ and from 1,300-450 cm⁻¹, showing that the structural characteristics of the zeolites remain unaltered.

In conclusion, it is possible to verify that the biofilm of Arthrobacter viscosus supported in X or Y zeolites presents a removal capacity of Cr ions contained in weak solutions and, therefore, they are a suitable solution for usage in bio-remediation.

The reduction of Cr (VI) to Cr (III) is promoted by the biofilm, being Cr (III) retained in the zeolite by ion exchange.

### REFERENCES

[1] S. E. Bailey, T. J. Olin, R. M. Bricka and D. D. Adrian, Water Res., Vol. 33, No. 11 (1999) 2469.
[2] E. Erdem, N. Karapinar and R. Donat, J. Coll. Inter. Sci., 280 (2004) 309.
[3] S. K. Pitcher, R. C. T. Slades and N. I. Ward, Sci. Total Environ., 334-335 (2004) 161.
[4] A. Corma and H. Garcia, Eur. J. Inorg. Chem. (2004) 1143.
[5] S. Babel and T. A. Kurniawan, J. Haz. Mat., B97 (2003) 219.
[6] J. R. Duncan, D. Brady and A. Stoll, Environ. Technol. No. 15 (1994) 429.
[7] G. M. Woodburn, Q. Yu and J. T. Matheickal, Water Res., 32 (1999) 400.
[8] C. Quintelas and T. Tavares, Biotechnol. Letters, Vol. 23 (2001) 1349.
[9] Y. T. Wang and H. Shen, Water Res., 7 (1997) 727.

1. Biosorption system produced from biofilms supported in faujasite (FAU) zeolites, characterized by comprising biofilms of Arthrobacter viscosus bacterium supported on faujasite (FAU) zeolites.

2. Process for obtaining the biosorption system, according to claim 1, characterized by making a bacterial culture of Arthrobacter viscosus grow in the presence of pre-treated FAU zeolites.

3. Process for obtaining the biosorption system, according to claim 2, characterized by performing a pre-treatment of the FAU zeolites, by their calcination at 500° C. during 6 hours under a dry air stream.

4. Process for obtaining the biosorption system according to claim 2, characterized by comprising the growth of the Arthrobacter viscosus bacterium in the presence of the pre-treated FAU zeolites and in an adjusted and sterilized growth medium at 120° C. during 20 min.

5. Process for obtaining the biosorption system according to claim 2, characterized by comprising a recovery step, of the biomass fraction loaded with chromium for further reutilization in a biosorption system, after the hexavalent chromium solutions having contacted the biosorbents of this system.

6. Process for obtaining the biosorption system according to claim 5, characterized by the recovery step of the biomass fraction loaded with chromium being performed by centrifugation of the solid matrix, containing the referred ions, at 5000 rpm and subsequent removal of organic material from Arthrobacter viscosus bacterium, by calcination at 500° C. during 6 hours under a dry air stream.

7. Use of the biosorption system, according to claim 1 characterized by being applicable in industrial, mining and agriculture wastewater treatment, for hexavalent chromium removal.

8. Use of the biosorption system, according to claim 7, characterized by comprising the contact of the system with hexavalent chromium solutions present in the wastewaters to be treated.

9. Use of the biosorption system, according to claim 8, characterized by comprising the contact of the system, with hexavalent chromium solutions with concentrations between 50 and 250 mg_{Cr}/L.

10. Use of the biosorption system, according to claim 5, characterized by the contact between the system and with hexavalent chromium solutions being performed at a temperature of 28° C., in medium stirring.
